(19) United States
(12) Patent Application Publication
(10) Pub. No.: US 2007/0083109 A1
(43) Pub. Date: Apr. 12, 2007

(54) Adaptive line synthesis for ultrasound

(76) Inventors: Kutay F. Ustuner, Mountain View, CA (US); D-L Donald Liu, Issaquah, WA (US); Lewis J. Thomas, Palo Alto, CA (US); Charles E. Bradley, Burlingame, CA (US); Anming He Cai, San Jose, CA (US); Robert Nolen Phelps, Fall City, WA (US); John C. Lazenby, Fall City, WA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/238,889

(22) Filed: Sep. 28, 2005

(51) Int. Cl. A61B 8/00

(52) U.S. Cl. 600/437

(57) ABSTRACT

Adaptive line synthesis is provided. Line synthesis of collinear receive beams responsive to spatially distinct transmit beams is a function of many parameters, such as spatial or temporal frequency response of one or more of the receive beams, synthesis function, number of receive beams synthesized, or acquisition sequence. One or more of these parameters is set or adapts as a function of processor estimated or user provided information. By adapting the line synthesis, the performance and image quality is optimized as appropriate for the received data or desired imaging, such as detail resolution, contrast resolution, temporal resolution, shift-invariance and penetration.

### BACKGROUND

This present invention relates to coherent combinations of received ultrasound signals. In particular, adaptive line synthesis is provided for ultrasound.

Line synthesis is a coherent image formation technique where multiple collinear receive beams, each formed in response to a spatially distinct transmit beam, are combined prior to amplitude detection. A spatially distinct transmit beam insonifies a region of interest at a unique angle. Line synthesis may allow formation of high-quality high-frame rate images using receive multibeam (receive beams formed in parallel using data acquired in response to a transmit event). Line synthesis may improve lateral resolution and uniformity of images formed using receive multibeam and lower clutter due to aberration.

The Sequoia ultrasound system uses line synthesis, such as disclosed in U.S. Pat. No. 5,623,928, the disclosure of which is incorporated herein by reference. Focused transmit beams scan a field of view. A two-beam receive multibeam is formed at each transmit event such that each receive multibeam overlaps with the adjacent ones by one beam. Lines are synthesized by averaging the collinear receive beams of adjacent transmit events (i.e., line synthesis). Further lines are synthesized by averaging beams of each receive multibeam (i.e., beam interpolation). In the commercial product, the line synthesis is performed between two collinear receive beams or not performed. Different aspects of the line synthesis for combining the collinear receive beams were responsive to user inputs, such as the selection of a transducer, frequency, Space/Time™ control setting or other imaging characteristics.

Ustuner et al. described a high frame rate, high spatial bandwidth method using receive multibeam in U.S. Pat. No 6,309,356, the disclosure of which is incorporated herein by reference. Multiple transmit events, each with a weakly defocused, unfocused or weakly focused transmit beam and a distinct steering angle, insonify a large patch of a field of view. An N-beam receive multibeam is formed for each transmit event. Lines are synthesized by combining collinear receive beams that are formed in response to transmit events with distinct steering angles.

### BRIEF SUMMARY

By way of introduction, the preferred embodiments described below include a method, instructions and systems for adaptive line synthesis. Line synthesis of collinear receive beams responsive to spatially distinct transmit beams is a function of many parameters, such as spatial or temporal frequency response of one or more of the receive beams, synthesis function, number of receive beams synthesized, or acquisition sequence. One or more of these parameters is set or adapts as a function of processor estimated or user provided information. By adapting the line synthesis, the performance and image quality is optimized as appropriate for the received data or desired imaging, such as detail resolution, contrast resolution, temporal resolution, shift-invariance and penetration.

In first aspect, a method adapts ultrasound processing. Received signals for collinear receive beams responsive to spatially distinct transmissions are coherently combined. A processor estimates as a function of received ultrasound information. At least one parameter associated with the coherent combination is varied as a function of an output of the estimating.

In a second aspect, a computer readable storage medium has stored therein data representing instructions executable by a programmed processor for adaptive ultrasound processing. The instructions are for outputting information as a function of received ultrasound signals, varying at least one line synthesis parameter as a function of the information, and synthesizing collinear receive beams responsive to spatially distinct transmit beams, the synthesis being a function of the varied line synthesis parameter.

In a third aspect, a method adapts ultrasound processing. Received signals for at least three collinear receive beams are combined coherently. At least two of the at least three collinear receive beams are responsive to spatially distinct transmissions. At least one parameter associated with the coherent combination is varied as a function of user input.

In a fourth aspect, a computer readable storage medium has stored therein data representing instructions executable by a programmed processor for adaptive ultrasound processing. The instructions are for setting at least one line synthesis parameter as a function of user input, and synthesizing three or more collinear receive beams responsive to at least two spatially distinct transmit beams. The synthesis is a function of the set line synthesis parameter.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of one embodiment of a method for adaptive line synthesis ultrasound processing;

FIG. 2 is a graphical representation of one embodiment of transmit and receive beam interrelationships; and

FIG. 3 is a block diagram of one embodiment of a system for adaptive line synthesis ultrasound processing.

### DETAILED DESCRIPTION OF THE DRAWINGS AND PRESENTLY PREFERRED EMBODIMENTS

Line synthesis parameters adapt as a function of user input, amount of motion, acoustic clutter or electronic noise. Line synthesis parameters include the number, relative weighting, spatial and temporal frequency responses of the component or collinear receive beams. By varying synthesis parameters, the system can be optimized for best performance in either one or more of the following image quality aspects: detail resolution, contrast resolution, temporal resolution, shift-invariance and penetration.

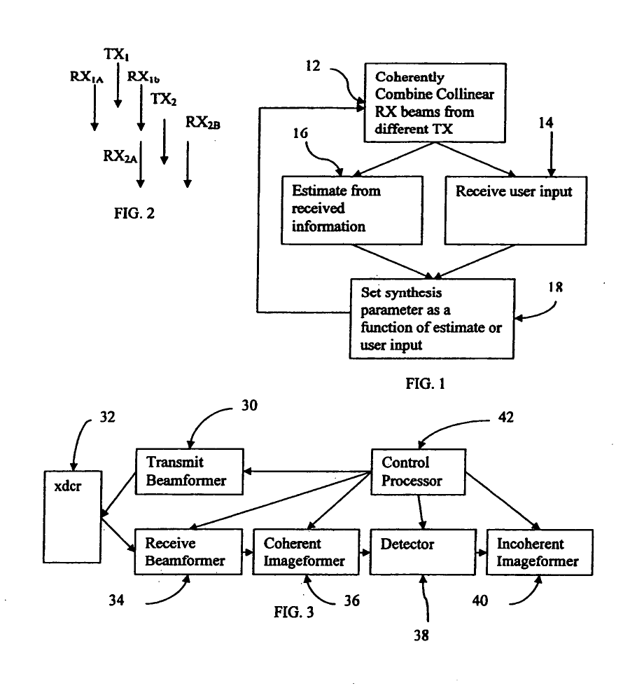

FIG. 1 shows a flow chart for a method for adaptive ultrasound processing. The method is implemented by or on the system of FIG. 3 or a different system. Additional, different or fewer acts may be provided. For example, the method does not include act 14. As another example, the method does not include act 16. The flow chart shows the variation of one or more parameters in act 18 occurring after combination in act 12. Alternatively, the adaptive parameters are determined prior to any combination in act 12.

In act 12, received signals for two, three or more collinear receive beams are coherently combined. Each or different sets of the collinear receive beams are responsive to spatially distinct transmissions. For example, with three or more different collinear receive beams, at least two of the at least three collinear receive beams are responsive to spatially distinct transmissions. Coherent combination synthesizes the collinear receive beams prior to detection. The synthesis is a function of line synthesis parameters.

Referring to FIG. 2, multiple noncollinear receive beams (RX_{1A} and RX_{1B}, and RX_{2A} and RX_{2B}) are formed in parallel or substantially simultaneously in response to each transmit firing (TX₁ and TX₂, respectively). The set of spatially distinct beams formed in parallel is called noncollinear multibeam or multibeam. As the number of beams in a multibeam increases (e.g., three or more), the transmit beam is wider to adequately insonify the locations of the receive beams. The wider transmit beam causes a decrease in resolution, increase in artifacts and decrease in signal-to-noise ratio (SNR). With receive multibeam, lateral resolution is limited to the one-way receive only resolution due to lack of or weak transmit focusing. The acoustic clutter is high and therefore contrast resolution is limited in the presence of aberration due to lack of redundancy. Redundancy at a particular spatial frequency is defmed as the attribute of having more than one transmit-receive element pair contributing to that spatial frequency. Image uniformity is compromised and the image becomes shift-variant due to lateral nonuniformity of the transmit main lobe.

In the example of FIG. 2, one receive beam (RX_{2A}) from one transmit event is collinear with another receive beam (RX_{1B}) from another transmit event. The line synthesis of the collinear receive beams may improve resolution and reduce artifacts. The lateral resolution to the confocal (two-way) equivalent lateral resolution is doubled since it is effectively a transmit synthetic aperture technique. Contrast resolution is improved in the presence of aberration by adding redundancy through the spatially distinct transmit beams. Image uniformity is improved by reducing shift variance.

At each transmit event (e.g., TX₁ or TX₂), the transmit beamformer sends a single beam, or multiple beams in parallel. FIG. 2 shows two transmit beams generated at different times. Each transmit beam is focused (i.e., converging wavefront), unfocused (planar wavefront) or defocused (diverging wavefront) and propagates along a particular nominal transmit beam axis or transmit line. The transmit beams formed in parallel may be collinear (share the same transmit line), or noncollinear. The collinear transmit beams formed in parallel or substantially simultaneously (collinear transmit multibeam) may differ in one or more of the transmit beamforming and pulse shaping parameters, such as focal depth, center frequency, apodization type, aperture width, bandwidth or other transmit beam characteristic. Additionally, different pulse codes (e.g., Barker, Golay, Hadamard codes or other orthogonal complementary code sets) can be transmitted simultaneously, and the received signals are decoded to separate out the signals corresponding to each transmit beam. Beams of a noncollinear transmit multibeam may have one or more distinct transmit beamforming or pulse shaping parameters, in addition to having distinct transmit lines.

At each receive event, the receive beamformer receives echoes from the object, and forms a beam or multiple beams in parallel. FIG. 2 shows two spatially distinct transmit events, and two receive beams formed in parallel or substantially simultaneously with each other in response to each transmit event. Three or more receive beams may be formed, including with or without a receive beam along the transmit line or collinear with the transmit beam. Each receive beam is dynamically focused along a particular nominal receive beam axis or receive line. The receive beams formed in parallel may be collinear (share the same receive line) or noncollinear. The collinear receive beams formed in parallel (collinear receive multibeam) may differ in one or more of the receive beamforming or pulse shaping parameters, such as the aperture center, aperture width, apodization type, center frequency, bandwidth or other receive beam characteristics. The noncollinear beams of a receive multibeam have different delays profiles. The remaining receive beamforming or echo shaping parameters such as aperture center, aperture width, apodization type, receive filter center frequency, bandwidth and spectral shape, may be the same or different. As an alternative to receive beamforming in a time domain, the receive beams may be formed in a frequency domain, such as disclosed in U.S. Pat. No. 6,685,641, the disclosure of which is incorporated herein by reference.

The transmit and corresponding receive events are repeated to sample the object or region in space, in time and/or in a parameter space. To sample the object in space, events with noncollinear transmit and receive beams or events with noncollinear receive beams are used. To sample the object in time, collinear events with identical beamforming and pulse shaping parameters are used. For example, for each Color Flow Mode line, multiple collinear events uniformly distributed in time are used. To sample the object in a parameter space, multiple collinear or noncollinear events with at least one distinct beamforming or pulse shaping parameter are used. As an example, noncollinear receive events are used with distinct transmit lines where at least one receive beam for one transmit event is collinear with at least one receive beam for another transmit event.

These space, time and parameter samples of the object are then processed and combined by the imageformer to form a frame or volume of image, or images. The line synthesis of act 12 is a coherent image formation technique where multiple collinear receive beams, each formed in response to a spatially distinct transmit beam, are combined prior to amplitude detection. The collinear receive beams combined to form a particular synthetic line are referred to as component beams. Component beams are combined by a synthesis function. The synthesis or combination function may be a simple summation or a weighted summation operation, but other functions may be used. The synthesis function includes linear or nonlinear functions and functions with real or complex, spatially invariant or variant component beam weighting coefficients. Nonlinear synthesis functions also include products, sum of powers with signs preserved such as:
O₁=Σₙwₙsgn (Iₙ)|Iₙ)|^{p}O=sgn (O₁)^{p}\sqrt{|O₁|}
where, Iₙ are the inputs and O is the final output. p=1 corresponds to linear synthesis. Nonlinear functions may also be implemented as arbitrary multi-input single-output maps.

The line synthesis is adaptive. One or more different parameters are set in act 18 based on information from acts 14 and/or 16.

In act 16, a processor adaptively responds to received ultrasound information. For example, a processor estimates a line synthesis parameter as a function of received ultrasound information. The received ultrasound information is the line synthesized information output in act 12 or other information. For example, the processor estimates the parameter from component beams, different combinations of receive beams or ultrasound information responsive to different scans. The adaptation occurs substantially constantly, periodically, in response to a trigger event (e.g., heart cycle event, user activation, or detection of another event). The processor outputs the parameter for controlling line synthesis, a control instruction for controlling the line synthesis, or other data used by another processor to control line synthesis.

The processor estimates an amount of motion, an aberration, a signal-to-noise ratio, combinations thereof or other characteristics of the scanned region or signals responsive to the scanned region. The optimum values of the line synthesis parameters may depend on the object being imaged. Various line synthesis parameters are varied or set as a function of motion, aberration and/or SNR.

The amount of motion is an amount of an object's motion relative to the transducer. For example, the amount of motion is of the heart or other organ or an amount of flow. The motion is relative to the transducer for the transmit and receive events with or without accounting for intentional or unintentional transducer motion relative to the patient. Temporal cross correlation, difference of sum or difference of samples along a line, in an area or in a volume estimates the motion. Other now known or later developed indication of motion may be used, such as an average Doppler tissue motion value as a function of time.

The processor estimates an amount of aberration by measuring the side lobe clutter energy. The aberration estimator may use the coherence factor, such as the coherence factor described in U.S. Pat. No. ______ (Publication No. ______ (application Ser. No. 10/814,959) (Attorney Docket No. 2004P01660US), the disclosure of which is incorporated herein by reference. The coherence factor indicates an amount of coherence of received data across the receive aperture. High coherence indicates little aberration, and low coherence indicates a larger aberration effect. In one embodiment, the coherence factor is calculated as a ratio of a coherent sum to an incoherent sum. In another embodiment, the coherence factor is derived from the spectrum of the aperture data, such as the ratio of the spectral energy within a pre-specified low frequency region to the total energy. In another embodiment, the coherence factor is the amplitude of the coherent sum. In yet another embodiment, the coherence factor for each spatial location in an image region is calculated as a function of different aperture sizes or other variables. Alternatively, the energy of received beams that fall substantially outside the geometric shadow of the transmit beam relative to the energy of received beams that fall substantially within the transmit beam indicate the coherence factor.

The processor estimates SNR or penetration by measuring the signal-to-mean noise ratio. The mean noise may be estimated by low pass filtering an image captured with the transmitters turned off. The signal information is obtained using the current imaging settings, but other imaging settings may be used.

In act 14, the line synthesis parameters adapt to user indications of preference or imaging characteristics. The optimum values of the line synthesis parameters depend on the relative priority of image quality aspects: detail resolution, contrast resolution, temporal resolution, image uniformity or SNR. Selection of a particular clinical application may indicate a priority. For example, a cardiac imaging application indicates a priority for temporal resolution. As another example, a fetal heart application in OB indicates a priority for temporal resolution (e.g., fewer component beams) as compared to general imaging of a fetus. In act 14, user input is received to determine the priorities.

The priorities are determined based on user selections, settings or other input. For example, the selection of a transducer may indicate a priority. Different transducers are used for different types of imaging or applications. If the same transducer is used for multiple applications with different priorities, the user selection of an application associated with the transducer indicates the priorities. As another example, the user inputs an indication of relative priority between spatial and temporal resolution. A separate user control dedicated to spatial and temporal resolution, a combination of settings, or selection of an application indicates this selection. Other user input indicating a user selected filtering setting may be used. For example, the user is able to select different types of filters or filtering characteristics (e.g., selecting between low pass smoothing or edge enhanced processing). As another example, the user selects an imaging frequency or frequencies. Frequency indicates relative priority of spatial resolution and penetration.

In act 18, a line synthesis parameter is set as a function of the output of acts 14 and/or 16. A processor sets or varies (i.e., resets) one or more line synthesis parameters as a function of processor estimated characteristic of the scanned region from received ultrasound data and/or as a function of user input indication of priority. One or more line synthesis parameters adapt as a function of user input or received data.

The number of the collinear receive or component beams is set. The number of component beams is increased if the image uniformity and shift-invariance is a priority. A greater number of component beams reduces a lack of uniformity due to averaging. The number of component beams and the angular separation of transmit events are adapted to achieve optimal imaging performance depending on the degree of motion and aberration. If motion is significant, such as in cardiac imaging or in a survey mode where the transducer is translated or rotated, only a small number of component beams may be synthesized without performing motion compensation. In order to attain maximum spatial (lateral) resolution with the minimum number of component beams, the angular separation of transmit events is increased until spectral overlap of the component beams is minimized without introducing a discontinuity in the synthesized spectrum. On the other hand, if motion is minimal and aberration is significant, such as in breast imaging, then a larger number of component beams with reduced angular separation can be synthesized to gain redundancy and suppress clutter caused by aberration.

The temporal frequency response of at least one of the collinear receive beams is set. The pulse repetition interval between component beams is minimized for increased temporal frequency response. Where temporal frequency response has less priority, the pulse repetition interval is increased, such as described above for dealing with aberration.

The spatial response of at least one of the collinear receive beams is set. Component image or beam spatial frequency response is a function of transmit angle, transmit focus depth (negative if virtual point source or plane wave), transmit apodization and receive apodization. For example if the detail and temporal resolution are the highest priorities, the transmit beam width and the angular separation of the transmit events are maximized and a more uniform receive apodization is selected. Maximizing lateral resolution requires only two component beams and a uniform receive apodization. The receive f-number is a function of the angular separation of the component beam transmit events to ensure continuity within the synthesized spectrum pass-band. In sector or Vector® scans, the transmit beams are formed along transmit lines at different angles. The spacing between transmit lines provides angular separation.

If contrast resolution is a high priority, the receive apodization is tapered, and the angular separation of transmit events is reduced accordingly to provide a smooth synthesized spectrum. Achieving good contrast resolution and lateral resolution at the same time is done by increasing the number of component beams while tapering the receive apodization at the edges. The redundancy at a given spatial frequency is given by the number of component beams that contribute to that particular spatial frequency. Increasing redundancy decreases sensitivity to aberration, whether aberration is due to tissue's speed of sound or attenuation nonuniformities, or transducer's element-to-element or system's channel-to-channel delay and amplitude variations. The receive apodization is also varied depending on aberration. If aberration is significant, a uniform apodization for component beams increases overlap of the component beam spectra and thus maximizes redundancy. If aberration is not significant, but contrast resolution (low side lobes) is a priority, the receive apodization is chosen so that the synthesized aperture function (i.e., synthesis of the two-way aperture functions for each component beam) has no discontinuity and has continuous derivatives up to a high order. For example, a uniform (box-car) aperture function has a discontinuity at its edges. A triangular aperture function, on the other hand, has no discontinuity itself but it has discontinuities in its first spatial derivative

The transmit focal depth is set as a function of SNR. If SNR is not sufficient, a negative focal depth (virtual point source) is moved away from the transducer to a larger negative offset, reducing the amount of divergence, or a positive focal depth is moved closer to the depth of interest, increasing the amount of focusing. This effectively reduces the size of the object insonified (FOV) per transmit event. The angular separation of the transmit events and the number of receive beams per receive multibeam are reduced accordingly. Alternatively, complimentary code sets code the temporal and/or spatial response of the component beams.

A combining function for the line synthesis is set. Synthesis combination functions include sum, weighted sum, linear, nonlinear, real weights, complex weights, spatially invariant, spatially variant, map-based, or other functions. The synthesis function adapts to the user or object. Different functions provide different priorities.

An acquisition sequence associated with the collinear receive beams is set. The component image data acquisition sequence is set as a function of an amount of motion. For example in three- or four-dimensional imaging, plane-by-plane, box-by-box, an amount of zigzagging or other sequence arrangement for scanning the volume varies. Greater motion dictates less time between transmit events associated with line synthesis to avoid motion artifacts.

As discussed above in examples, combinations of different parameters are set. A plurality of parameter sets of the parameters are available in one embodiment. One of the sets is selected based on the combination of outputs from acts 14 and/or 16 or based on a single output. Alternatively, a processor calculates the parameters based on the outputs. In another embodiment, a sub-set of one or more of the parameters is varied or set in response to the outputs and other parameters are maintained at a predetermined value.

FIG. 3 shows one embodiment of a system for adaptive ultrasound imaging. The system is an ultrasound imaging system, but other imaging systems using multiple transmit or receive antennas (i.e., elements) may be used. The system includes a transducer 32, a transmit beamformer 30, a receive beamformer 34, a coherent imageformer 36, a detector 38, an incoherent imageformer 40 and a control processor 42. Additional, different or fewer components may be provided, such as the system with a scan converter and/or display.

The transducer 32 is an array of a plurality of elements. The elements are piezoelectric or capacitive membrane elements. The array is configured as a one-dimensional array, a two-dimensional array, a 1.5D array, a 1.25D array, a 1.75D array, an annular array, a multidimensional array, combinations thereof or any other now known or later developed array. The transducer elements transduce between acoustic and electric energies. The transducer 32 connects with the transmit beamformer 30 and the receive beamformer 34 through a transmit/receive switch, but separate connections may be used in other embodiments.

Two different beamformers are shown in the system 10, a transmit beamformer 30 and the receive beamformer 34. While shown separately, the transmit and receive beamformers 30, 34 may be provided with some or all components in common. Both beamformers connect with the transducer 32. The transmit beamformer 30 is a processor, delay, filter, waveform generator, memory, phase rotator, digital-to-analog converter, amplifier, combinations thereof or any other now known or later developed transmit beamformer components. In one embodiment, the transmit beamformer 30 is the transmit beamformer disclosed in U.S. Pat. No. 5,675,554, the disclosure of which is incorporated herein by reference. The transmit beamformer is configured as a plurality of channels for generating electrical signals of a transmit waveform for each element of a transmit aperture on the transducer 32. The waveforms have relative delay or phasing and amplitude for focusing the acoustic energy. The transmit beamformer 30 includes a controller for altering an aperture (e.g. the number of active elements), an apodization profile across the plurality of channels, a delay profile across the plurality of channels, a phase profile across the plurality of channels and combinations thereof. A scan line focus is generated based on these beamforming parameters.

The receive beamformer 34 is a preamplifier, filter, phase rotator, delay, summer, base band filter, processor, buffers, memory, combinations thereof or other now known or later developed receive beamformer components. In one embodiment, the receive beamformer is one disclosed in U.S. Pat. Nos. 5,555,534 and 5,685,308, the disclosures of which are incorporated herein by reference. The receive beamformer 34 is configured into a plurality of channels for receiving electrical signals representing echoes or acoustic energy impinging on the transducer 32. Beamforming parameters including a receive aperture (e.g., the number of elements and which elements are used for receive processing), the apodization profile, a delay profile, a phase profile and combinations thereof are applied to the receive signals for receive beamforming. For example, relative delays and amplitudes or apodization focus the acoustic energy along one or more scan lines. A control processor controls the various beamforming parameters for receive beam formation. Beamformer parameters for the receive beamformer 34 are the same or different than the transmit beamformer 30.

Receive beamformer delayed or phase rotated base band data for each channel is provided to a buffer. The buffer is a memory, such as a first in, first out memory or a corner turning memory. The memory is sufficient to store digital samples of the receive beamformer 34 across all or a portion of the receive aperture from a given range. The beamformer parameters used by the transmit beamformer 30, the receive beamformer 34, or both are set for line synthesis. The beamformer parameters may be used as line synthesis parameters for forming the component beams.

The receive beamformer 34 includes one or more digital or analog summers operable to combine data from different channels of the receive aperture to form—one or a plurality of receive beams. Cascaded summers or a single summer may be used. In one embodiment, the beamform summer is operable to sum in-phase and quadrature channel data in a complex manner such that phase information is maintained for the formed beam. Alternatively, the beamform summer sums data amplitudes or intensities without maintaining the phase information.

The coherent imageformer processor 36 is a general processor, digital signal processor, control processor, application specific integrated circuit, digital circuit, digital signal processor, analog circuit, combinations thereof or other now known or later developed processors for performing line synthesis. In one embodiment, the coherent imageformer 36 is part of the receive beamformer 34 or control processor 36, but a separate or dedicated processor or circuit may be used in other embodiments. The coherent imageformer 36 includes memory buffers, complex multipliers and complex summers, but other components may be used.

The coherent imageformer 36 is operable to synthesize lines as a function of adaptive parameters. For example, the coherent imageformer 36 is operable to form data representing a range of depths or lateral locations from sequential component collinear beams or combine data from different sub apertures to form one or more lines of collinear data. Ultrasound lines are formed from receive beams formed by the receive beamformer 34. The synthesis may involve inter-beam phase correction as a first step. Multiple stages or parallel processing may be used to increase the throughput or number of receive beams processed for real-time imaging, such as associated with three- or four-dimensional imaging. The synthesis then combines the phase corrected beams through a coherent (i.e., phase sensitive) filter to form synthesized ultrasound lines.

In one embodiment, the coherent imageformer 36 includes pre-detection axial filtering for receive pulse shaping and decoding, phase correction to phase align receive beams in one or both of the lateral axes, and beam- and range-dependent gain for spatial weighting and/or masking of beams (i.e., weighting receive beams outside a transmit beam region with a zero, such as for plane wave transmissions with a sector or Vector® receive format). Collinear receive beams are combined for line synthesis after any phase correction. The combination is prior to detection or coherent. Any combination function may be used, such as summation, weighted summation or nonlinear combination of collinear receive beams formed at distinct transmit events. The line synthesis is of receive beams responsive to transmit beams along same or different scan lines. For example, the line synthesis is for phase inversion (receive beams associated with transmissions with different, such as opposite, phases), contrast pulse sequences (receive beams associated with transmissions at different amplitudes and/or phases), color flow, transmit focus synthesis (receive beams associated with transmissions to different focal depths), or other image forming processes coherently combining collinear receive beams from distinct transmissions along a same scan line. As another example, the line synthesis is for combination of collinear receive beams formed in response to distinct noncollinear transmit events.

Additional, different or fewer components and associated functions may be provided by the coherent image former 36. Analytic beam interpolation forms new lines of data between receive beams from the same transmissions (e.g., RX_{1A} combined with RX_{1B} to form an analytic beam, such as along the scan line for TX₁. Analytic beams may increase the lateral sampling rate to prevent aliasing due to noncollinear event synthesis. Pre-detection lateral filtering provides lateral whitening or artifact reduction. Analytic line interpolation forms new lines of data between synthesized lines. Analytic line interpolation may increase the lateral sampling rate to prevent aliasing due to envelope detection.

The detector 38 is a general processor, digital signal processor, control processor, application specific integrated circuit, digital circuit, digital signal processor, analog circuit, combinations thereof or other now known or later developed processors for envelope detection. The detector 38 detects any of various characteristics, such as amplitude, intensity (i.e., amplitude squared) or log-compressed amplitude. A log compressor is provided in one embodiment, but may alternatively be positioned after the incoherent imageformer 40. In alternative embodiments, Doppler or flow detection is provided.

The incoherent imageformer 40 is operable on detected data to combine incoherently multiple ultrasound lines. In one embodiment, the input to the incoherent imageformer 40 is the intensity data, and, in another, the input is the log-compressed data. The ultrasound lines combined may have differing temporal spectra or differing spatial spectra. Sequential focus stitching (e.g., zone cross-fade) may be performed in addition to frequency and spatial compounding. Any extra transmit events that are not synthesized coherently may be combined incoherently or compounded to reduce speckle and improve image uniformity.

In one embodiment, the incoherent imageformer 40 includes buffers, filters, summers, multipliers, processors or other components for implementing the compounding and/or other incoherent processes. For example, the incoherent imageformer 40 performs post-detection (video) axial filtering for receive pulse shaping, collinear multibeam spatial and/or frequency compounding, collinear transmit event compounding of corresponding collinear receive beams for transmit/receive frequency compounding, sequential focus, transmit focus compounding, or other purposes, noncollinear transmit event compounding of collinear receive beams for transmit/receive spatial compounding, post-detection lateral video filtering for lateral response shaping or artifact reduction, and adaptive gain, compression and mapping. Different, fewer or additional incoherent processes may be provided.

In one embodiment, each coherent image former 36 and each incoherent imageformer 40 are operable for a limited number of channels, such as a group of 16 channels. A plurality of devices is provided for each group of channels. The outputs may then be used to synthesize further data or provide further incoherent combinations. In one embodiment, the incoherent imageformer 40 is provided with a feedback from the detector 38 for compounding detected data.

The images or receive beams combined coherently or incoherently are on a same acoustic or scan grid. Alternatively, a spatial transformation or scan conversion aligns the component beams or associated images. The data is output as an one-, two-, or three-dimensional representation on the display. Other processes, such as the generation of text or graphics may also be performed for generating an image on a display. For example, a display dynamic range is set, filtering in space and time using a linear or nonlinear filter which may be an FIR or IR filter or table-based is provided, and/or the signal amplitude is mapped to display values as a function of a linear or non-linear map. The non-linear map may use any of various inputs, such as both filtered and unfiltered versions of the data being input in selecting a corresponding brightness. Data optimized for contrast may be input with the same or similar data optimized for spatial resolution. The input data is then used to select brightness or display intensity.

As part of the image forming process, the control processor 42 sets a scan pattern or acquisition sequence, number of simultaneous receive beams, a number of sequential beams, a number of sub apertures, a number of focal zones in a same scan line, a number of component beams compounded together, receive multiple beam parameters, combination function, component beam temporal frequency response, component beam spatial frequency response, combinations thereof or other now known or later developed parameters for coherent combination by the coherent imageformer 36. The parameters are set as a function of received ultrasound data and/or user input. The received ultrasound data is from any where along the processing path, such as from the receive beamformer 34, the coherent imageformer 36, the detector 38 or the incoherent detector 40. The received ultrasound data used to vary, adapt or set the parameters is also the data to be coherently combined or is different data, such as associated with different transmit events. The user input is provided from an input device directly to the control processor 42 or is routed from another processor.

The instructions for implementing the adaptive processes, methods and/or techniques discussed above are provided on computer-readable storage media or memories, such as a cache, buffer, RAM, removable media, hard drive or other computer readable storage media. The instructions are implemented on a single device, such as the control processor 42, or a plurality of devices in a distributed manner. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, filmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for adaptive ultrasound processing, the method comprising:
combining coherently received signals for collinear receive beams responsive to spatially distinct transmissions;
estimating with a processor as a function of received ultrasound information; and
setting at least one parameter associated with the coherent combination as a function of an output of the estimating.

2. The method of claim 1 wherein combining coherently comprises line synthesis prior to detection.

3. The method of claim 1 wherein setting the at least one parameter comprises varying a number of the collinear receive beams.

4. The method of claim 1 wherein setting the at least one parameter comprises varying a temporal frequency response of at least one of the collinear receive beams.

5. The method of claim 1 wherein setting the at least one parameter comprises varying a spatial response of at least one of the collinear receive beams.

6. The method of claim 1 wherein setting the at least one parameter comprises varying a spatial response of at least one of the transmit beams.

7. The method of claim 6 wherein varying the spatial response of at least one of the transmit beams comprises varying the angle of the transmit beam.

8. The method of claim 6 wherein varying the spatial response of at least one of the transmit beams comprises varying the width of the transmit beam.

9. The method of claim 1 wherein setting the at least one parameter comprises varying a combining function for the combining act.

10. The method of claim 1 wherein setting the at least one parameter comprises varying an acquisition sequence associated with the receive beams.

11. The method of claim 1 wherein estimating comprises estimating an amount of motion.

12. The method of claim 1 wherein estimating comprises estimating an aberration.

13. The method of claim 1 wherein estimating comprises estimating a signal-to-noise ratio.

14. In a computer readable storage medium having stored therein data representing instructions executable by a programmed processor for adaptive ultrasound processing, the storage medium comprising instructions for:
outputting information as a function of received ultrasound signals;
setting at least one line synthesis parameter as a function of the information; and
synthesizing collinear receive beams responsive to spatially distinct transmit beams, the synthesis being a function of the varied line synthesis parameter.

15. The instructions of claim 14 wherein setting the at least one line synthesis parameter comprises varying a number of the collinear receive beams, a temporal frequency response of at least one of the collinear receive beams, a spatial response of at least one of the collinear receive beams, a synthesis function for the synthesizing act, an acquisition sequence associated with the receive beams, or combinations thereof.

16. The instructions of claim 14 wherein outputting information as a function of received ultrasound signals comprises outputting the information responsive to an amount of motion, an aberration, a signal-to-noise ratio or combinations thereof.

17. A method for adaptive ultrasound processing, the method comprising:
forming a first receive multibeam with at least three spatially distinct beams using data from a first transmit event;
forming a second receive multibeam with at least three spatially distinct beams using data from a second spatially distinct transmit event, where at least one of the beams of the second receive multibeam is substantially collinear with a beam of the first receive multibeam;
combining coherently at least one of the said collinear receive beams; and
setting at least one parameter associated with the coherent combination as a function of user input.

18. The method of claim 17 wherein combining coherently comprises line synthesis prior to detection.

19. The method of claim 17 wherein setting the at least one parameter comprises varying a number of the collinear receive beams.

20. The method of claim 17 wherein setting the at least one parameter comprises varying a temporal frequency response of at least one of the collinear receive beams.

21. The method of claim 17 wherein setting the at least one parameter comprises varying a spatial response of at least one of the collinear receive beams.

22. The method of claim 17 wherein setting the at least one parameter comprises varying a combining function for the combining act.

23. The method of claim 17 wherein setting the at least one parameter comprises varying an acquisition sequence associated with the receive beams.

24. The method of claim 17 wherein setting as a function of user input comprises varying as a function of a transducer selection.

25. The method of claim 17 wherein setting as a function of user input comprises varying as a function of an imaging application selection.

26. The method of claim 17 wherein setting as a function of user input comprises varying as a function of user indication of relative priority between spatial and temporal resolution.

27. The method of claim 17 wherein setting as a function of user input comprises varying as a function of a user filtering setting.

28. The method of claim 17 wherein setting as a function of user input comprises varying as a function of a user frequency setting.

29. The method of claim 17 wherein setting as a function of user input comprises varying as a function of a user indication of priority.

30. In a computer readable storage medium having stored therein data representing instructions executable by a programmed processor for adaptive ultrasound processing, the storage medium comprising instructions for:
setting at least one line synthesis parameter as a function of user input; and
synthesizing three or more collinear receive beams responsive to at least two spatially distinct transmit beams, the synthesis being a function of the set line synthesis parameter.

31. The instructions of claim 30 wherein setting the at least one line synthesis parameter comprises varying a number of the collinear receive beams, a temporal frequency response of at least one of the collinear receive beams, a spatial response of at least one of the collinear receive beams, a synthesis function for the synthesizing act, an acquisition sequence associated with the receive beams, or combinations thereof.

32. The instructions of claim 30 wherein setting comprises setting as a function of a transducer selection, an imaging application selection, a user indication of relative priority between spatial and temporal resolution, a user filtering setting, a user frequency setting, a user indication of priority, or combinations thereof.
